(19) United States
(12) Patent Application Publication
(10) Pub. No.: US 2026/0083935 A1
(43) Pub. Date: Mar. 26, 2026

(54) RAPID EXCHANGE CATHETER WITH INTERLOCKING TRANSITION INTERFACE

(71) Applicant: Covidien LP, Mansfield, MA (US)

(72) Inventors: Danyong Zeng, Aliso Viejo, CA (US); Eanna P. Connolly, Galway (IE); Onnik E. Tchulluian, Costa Mesa, CA (US); Aran Murray, Galway (IE); Duncan Z. Ashby, Rancho Cucamonga, CA (US)

(21) Appl. No.: 19/340,967

(22) Filed: Sep. 26, 2025

Related U.S. Application Data
(60) Provisional application No. 63/699,569, filed on Sep. 26, 2024.

(51) Int. Cl. A61M 25/00; A61M 25/01; A61M 25/09

(52) U.S. Cl. CPC A61M 25/005; A61M 25/0021; A61M 25/09; A61M 2025/0024; A61M 2025/0183

(57) ABSTRACT

Rapid exchange catheters with interlocking transition interfaces are disclosed. An example catheter includes an elongate body having a distal end portion carrying an interventional element thereon and a proximal end portion. The elongate body includes a distal shaft having a receptacle and a proximal shaft having a semi-tubular portion that is inserted into the distal shaft receptacle. The semi-tubular portion includes an engagement feature that limits movement of the semi-tubular portion with respect to the distal shaft receptacle. The catheter further includes an exchange port along an intermediate portion of the elongate body, and a guidewire lumen extending from the exchange port to the distal end portion of the elongate body.

### CROSS-REFERENCE TO RELATED APPLICATIONS

This application claims the benefit of and priority to U.S. Provisional Patent Application Ser. No. 63/699,569 filed Sep. 26, 2024, the entire disclosure of which is incorporated by reference herein.

### TECHNICAL FIELD

The present technology relates generally to systems for treating lesions throughout the body within body lumens. Specifically, some embodiments of the present technology relate to balloon catheters having interlocking transition interfaces.

### BACKGROUND

There are many conditions in which a catheter is required to treat a lesion within a body lumen such as a blood vessel. For instance, balloon catheter procedures are commonly used for treating conditions such as myocardial ischemic attack (MIA) and intracranial atherosclerotic disease (ICAD). These conditions are commonly characterized by narrowed or blocked blood vessels. In balloon catheter procedures, the catheter is typically introduced through a remote region of a patient's body, such as the leg or arm. The catheter shaft is directed along a guidewire through the patient's vasculature, and the balloon, which is disposed on a distal end of the catheter, is inflated at a target site to exert a radial force on the lumen tissue. In some cases, a treatment device, such as a stent, may be deployed during inflation of the balloon to prevent further blockage within the lumen or restenosis. In the case of ICAD, a balloon may be used to open the vessel, followed by deployment of a stent to maintain vessel patency after the balloon is removed.

In such balloon catheter procedures, there are inherent risks in treating vascular lesions. A physician pushes the catheter shaft through a patient's vasculature to reach a target treatment site. In some cases, the target site is located deep within tortuous anatomy of the patient and therefore, precise navigation is required to avoid damage to tissue during insertion. The physician must transmit a sufficient pushing force from the proximal end of the catheter to the distal end where the balloon is located; due to the geometry of the catheter, the high push force compared to the small cross-section of the catheter normal to the force can lead to kinking along its length or other mechanical failures during pushing. Accordingly, it is desirable to provide a catheter with sufficient strength for transmitting force along the catheter length and sufficient flexibility for navigation, particularly around tortuous anatomy.

Once the catheter-mounted balloon is directed to the target site, the balloon is expanded within the body lumen. Inflation medium, typically saline or another fluid, is delivered through the catheter's inflation lumen from the proximal end to the distal end of the system. While the balloon inflates, some or all flow through the target site lumen is inhibited. Lack of blood flow to any region of the body is dangerous, as that region fails to receive necessary nutrients and oxygen without blood flow. This lack of blood flow can lead to cell death in those tissues (ischemia), which is particularly dangerous in neurovascular procedures where blood flow to cerebral vasculature would be inhibited, causing loss of function in brain tissue. Accordingly, there remains a need for improved balloon catheter systems that can navigate through tortuous anatomy while maintaining key performance parameters such as kink resistance and short inflation time.

### SUMMARY

The present technology is illustrated, for example, according to various aspects described below. Various examples of aspects of the present technology are described as numbered Examples (1, 2, 3, etc.) for convenience. These are provided as examples and do not limit the present technology. It is noted that any of the dependent clauses may be combined in any combination, and placed into a respective independent Example. The other Examples can be presented in a similar manner.

Example 1: A catheter comprising: a proximal shaft comprising: a tubular body defining a proximal inflation lumen therethrough; and a skived portion at a distal end region of the tubular body, the skived portion having first and second prongs; and a distal shaft carrying an inflatable member thereon, the distal shaft comprising: a distal inflation lumen having a distal end portion in fluid communication with the inflatable member; a guidewire lumen extending therethrough; and a transition interface including: a proximal opening of the guidewire lumen into which a guidewire can be slidably inserted; and a receptacle through which the skived portion of the proximal shaft tubular body is disposed such that the first and second prongs are placed in compression along a lateral direction and the prongs are interlocked with the receptacle, wherein the proximal inflation lumen is in fluid communication with the distal inflation lumen.

Example 2. The catheter of Example 1, wherein the skived portion is integrally formed with the tubular body.

Example 3. The catheter of Example 1 or Example 2, wherein each of the first and second prongs of the skived portion has a semi-annular cross-section.

Example 4. The catheter of any one of Examples 1-3, wherein the skived portion defines a distal taper such that an arc length of the skived portion is smaller in a distalmost segment of the skived portion than at a proximalmost segment of the skived portion.

Example 5. The catheter of any one of Examples 1-4, wherein the skived portion further comprises a lateral projection configured to abut a proximal face of the transition interface.

Example 6. The catheter of any one of Examples 1-5, wherein the tubular body comprises one or more flexibility-enhancing cuts along its length.

Example 7. The catheter of any one of Examples 1-6, further comprising a transition shaft having a distal end coupled to the distal shaft, the transition shaft circumferentially surrounding at least the skived portion of the proximal shaft tubular body.

Example 8. A catheter comprising: a proximal shaft comprising: a tubular body defining a proximal inflation lumen therethrough; a skived portion at a distal end region of the tubular body; and a lateral projection disposed along the skived portion; and a distal shaft carrying an inflatable member thereon, the distal shaft comprising: a distal inflation lumen having a distal end portion in fluid communication with the inflatable member; a guidewire lumen extending therethrough; and a transition interface including a proximal face that defines: a proximal opening of the guidewire lumen into which a guidewire can be slidably inserted; and a receptacle through which the skived portion of the proximal shaft tubular body is inserted such that the lateral projection disposed along the skived portion abuts the proximal face, wherein the proximal inflation lumen is in fluid communication with the distal inflation lumen.

Example 9. The catheter of Example 8, wherein the skived portion is integrally formed with the tubular body.

Example 10. The catheter of Example 8 or Example 9, wherein the skived portion has a semi-annular cross-section.

Example 11. The catheter of any one of Examples 8-10, wherein the skived portion defines a distal taper such that an arc length of the skived portion is smaller in a distalmost segment of the skived portion than at a proximalmost segment of the skived portion.

Example 12. The catheter of any one of Examples 8-11, wherein the skived portion further comprises first and second prongs.

Example 13. The catheter of any one of Examples 8-12, wherein the lateral projection is a first lateral projection extending along a first circumferential direction, the skived portion further comprising a second lateral projection extending along a second, opposite circumferential direction.

Example 14. The catheter of any one of Examples 8-13, wherein the lateral projection comprises a wing feature.

Example 15. The catheter of any one of Examples 8-14, wherein the proximal inflation lumen is in fluid communication with the distal inflation lumen via the receptacle.

Example 16. A catheter comprising: an elongate body having a distal end portion carrying an interventional element thereon and a proximal end portion, the elongate body comprising: a distal shaft having a receptacle; and a proximal shaft having a semi-tubular portion that is inserted into the distal shaft receptacle, wherein the semi-tubular portion includes an engagement feature that limits movement of the semi-tubular portion with respect to the distal shaft receptacle; an exchange port along an intermediate portion of the elongate body; and a guidewire lumen extending from the exchange port to the distal end portion of the elongate body.

Example 17. The catheter of Example 16, wherein the interventional element comprises an inflatable member, the catheter further comprising an inflation lumen extending from the elongate body proximal end portion to the elongate body distal end portion, wherein the inflation lumen is in fluid communication with the inflatable member.

Example 18. The catheter of Example 16 or Example 17, wherein the exchange port is disposed at the interface between the distal shaft and the proximal shaft.

Example 19. The catheter of any one of Examples 16-18, wherein the engagement feature comprises a first and second prongs along the semi-tubular portion.

Example 20. The catheter of any one of Examples 16-19, wherein the engagement feature comprises a lateral projection disposed along the semi-tubular portion.

Additional features and advantages of the present technology are described below, and in part will be apparent from the description, or may be learned by practice of the present technology. The advantages of the present technology will be realized and attained by the structure particularly pointed out in the written description and claims hereof as well as the appended drawings.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is a partial side section view of a catheter assembly in accordance with embodiments of the present technology.

FIG. 2 is a perspective exploded view of the catheter assembly showing the transition interface.

FIG. 3A is a top view of the proximal shaft of the catheter according to some embodiments of the present technology.

FIG. 3B is a side view of the proximal shaft of the catheter according to some embodiments of the present technology.

FIG. 3C is a bottom view of the proximal shaft of the catheter according to some embodiments of the present technology.

FIG. 4A is a perspective view of the catheter at the transition interface during assembly according to some embodiments of the present technology.

FIG. 4B is a cross-sectional view of the catheter at the transition interface along the line 4B-4B.

FIG. 5A is a perspective view of the proximal shaft of the catheter according to some embodiments of the present technology.

FIG. 5B is a top view of the proximal shaft of the catheter according to some embodiments of the present technology.

FIG. 5C is a side view of the proximal shaft of the catheter according to some embodiments of the present technology.

FIG. 6A is a perspective view of the catheter at the transition interface during assembly according to some embodiments of the present technology.

FIG. 6B is a cross-sectional section view of the catheter at the transition interface along the line 6B-6B.

### DETAILED DESCRIPTION

The present technology relates to endovascular systems, devices, and methods for treating lesions within body lumens, and associated devices and methods. In some implementations, a rapid-exchange balloon catheter can be used for treatment at an intravascular site, such as within the neurovasculature of a patient. Unlike traditional over-the-wire catheters, rapid-exchange catheters feature a shortened guidewire lumen that extends only through the distal portion of the catheter. This design allows for easier catheter exchange without the need for long exchange wires or extension wires, reducing procedure time and improving efficiency. Rapid-exchange catheters also offer enhanced pushability and trackability, making them easier to navigate through tortuous blood vessels. However, the design of a rapid-exchange catheter typically requires two separate elements to be joined together at a junction: a proximal shaft having an inflation lumen extending therethrough, and a distal shaft having both a distal inflation lumen and a guidewire lumen extending therethrough. The junction of these two shafts can present a challenge in achieving the desired mechanical properties of the catheter, such as column strength and kink resistance. In some cases, the junction creates a sharp discontinuity in the stiffness (or other mechanical properties) of the catheter, which may render the junction a point susceptible to failure (e.g., kinking or buckling) during advancement through the patient's blood vessel. This problem is particularly pronounced in the small, tortuous vessels of the neurovasculature.

Embodiments of the present technology provide a rapid-exchange balloon catheter with an interlocking transition between a proximal shaft and a distal shaft and addresses the shortcomings associated with conventional catheters. In some embodiments, for instance, a catheter includes a transition interface in which a guidewire can be slidably inserted into a proximal opening of a distal shaft, and a semi-tubular portion of a proximal shaft can be inserted into a receptacle in the distal shaft. The transition interface can additionally include an engagement feature on the proximal shaft that limits movement of the proximal shaft relative to the distal shaft. The proximal shaft may have a semi-annular skived portion that is slidably inserted into the receptacle of the distal shaft. In operation, the proximal shaft and the distal shaft are connected such that the proximal and distal inflation lumens are in fluid communication. The catheter system can include an interventional element, such as a balloon, carried on the distal end portion, to treat lesions within body lumens. In some embodiments, the catheter system additionally includes a treatment device, such as a stent, disposed on the outer surface of the interventional element, to be deployed upon operation of the catheter system for implantation within the body. A target body lumen can be treated such that inflation of the interventional element applies a radial force to the surrounding lumen tissue to expand the lumen's cross-sectional area and, upon inflation, a device can remain within the lumen to maintain lumen patency at the target site.

As noted above, the transition interface between the proximal shaft and the distal shaft can include an engagement feature that limits movement of the proximal shaft relative to the distal shaft and/or facilitates interlocking of these two components. In some embodiments, the engagement feature is defined by first and second prongs at the distal end portion of the proximal shaft which are placed in compression along a lateral direction within the receptacle of the distal shaft such that the prongs are interlocked with the receptacle in a fully assembled state. The compression in the lateral direction prohibits movement of the proximal shaft relative to the distal shaft when the proximal shaft is positioned at a specific location within the distal shaft receptacle. This location can be predetermined to be a position where shaft flexibility and pushability are optimized. In other embodiments, the engagement feature is defined by a lateral projection on the proximal shaft that abuts the proximal face of the distal shaft at the transition interface in a fully assembled state. Similar to the previously described pronged embodiments, the interface between the lateral projection and the proximal face of the distal shaft prohibits movement of the proximal shaft relative to the distal shaft when the proximal shaft is positioned at a location where the lateral projection abuts the proximal face. In still other embodiments, the engagement feature can include both first and second prongs of the proximal shaft to be placed in compression along a lateral direction within the receptacle of the distal shaft and a lateral projection on the proximal shaft that abuts the proximal face of the distal shaft.

The present technology can provide advantages compared to conventional catheter devices. The engagement feature of the proximal shaft at the transition interface facilitates catheter assembly to ensure proper orientation and positioning within the receptacle of the distal shaft. Proper assembly improves the performance of the catheter system, providing an optimal push efficiency, flexibility, lumen patency, and kink resistance. Embodiments of the present technology can include a direct interface between proximal and distal shafts. In contrast, conventional systems typically require additional components or features to facilitate a pushing force between proximal and distal shafts. As a result, navigation of the catheter through tortuous anatomy may be compromised due to a lack of flexibility at the transition interface. Previous systems that have attempted to incorporate solutions to increase flexibility and maintain push efficiency additionally increase manufacturing cost and complexity. Conventional catheter systems also face limitations with transition interface integrity. These systems typically require features that serve to connect the proximal and distal shafts, but inadvertently limit the inflation lumen patency at the transition interface. This interference can increase inflation time, which increases the amount of time that flow is restricted through the target body lumen. This problem is particularly prevalent in tortuous anatomical regions, such as in neurovascular procedures, and can quickly lead to ischemia in patients. Neurovascular regions have smaller, more tortuous lumens, but require similar mechanical properties in catheter systems for treating lesions. In some embodiments of the present technology, the transition interface is designed such that an inflation lumen patency is maintained to reduce inflation times. The transition interface can be configured such that an engagement feature defines a desirable insertion position that can be repeatably manufactured to maintain lumen patency, catheter stiffness, and flexibility. Further advantages will be made apparent with reference to embodiments of the present technology.

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

As used herein, the terms “vertical,” “lateral,” “upper,” and “lower” can refer to relative directions or positions of features of the embodiments disclosed herein in view of the orientation shown in the Figures. For example, “upper” or “uppermost” can refer to a feature positioned closer to the top of a page than another feature. These terms, however, should be construed broadly to include embodiments having other orientations, such as inverted or inclined orientations where top/bottom, over/under, above/below, up/down, and left/right can be interchanged depending on the orientation.

### I. OVERVIEW OF EXAMPLE TREATMENT SYSTEMS AND DEVICES

The present technology provides systems, devices, and methods for treating lesions within a vessel lumen. Although many of the embodiments are described below with respect to devices, systems, and methods for treating a narrowing of cerebral or intracranial vessels, other applications and other embodiments in addition to those described herein are within the scope of the technology. For example, the treatment systems and methods of the present technology may be used to within body lumens other than blood vessels (e.g., the digestive tract, etc.) and/or may be used to within blood vessels outside of the brain (e.g., pulmonary, abdominal, cervical, or thoracic blood vessels, or peripheral blood vessels including those within the legs or arms, etc.).

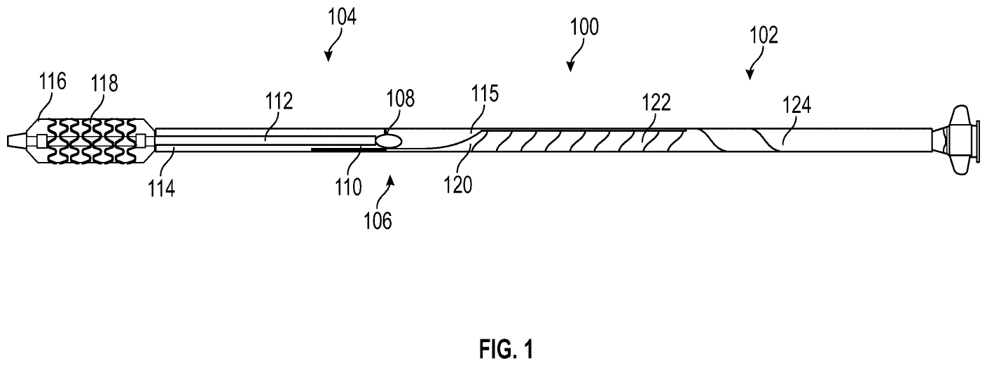

FIG. 1 is a partial side section view of a catheter assembly 100 in accordance with embodiments of the present technology. The catheter assembly 100 comprises a proximal shaft 102 and a distal shaft 104 joined at a transition interface 106, which together form an elongate body designed for intravascular navigation and treatment delivery.

The distal shaft 104, which is configured for placement within a patient's vasculature, carries an inflatable member 116 near its distal end. This inflatable member 116 may be a balloon used for angioplasty procedures or for expanding a treatment device. In some implementations, a treatment device 118, such as an expandable stent, may be disposed on the outer surface of the inflatable member 116. The stent may be a bare metal stent, a drug-eluting stent, or another type of implantable vascular prosthesis. In some variations, the inflatable member 116 may be omitted altogether. Additionally or alternatively, the treatment device can take other forms, such as electrodes carried by the catheter and configured to deliver electrical current to a target area, or any other suitable type of treatment device.

The distal shaft 104 includes a guidewire lumen 112 that extends throughout its length. This lumen 112 is designed to accommodate a guidewire, which is used to navigate the catheter through the vascular system to a target treatment site. The guidewire lumen 112 has a proximal opening 108 strategically located at the transition interface 106. This proximal opening 108 serves as a rapid-exchange port, allowing for easier and quicker exchange of guidewires or catheters without the need for extremely long guidewires or guidewire extensions.

In addition to the guidewire lumen 112, the distal shaft 104 includes a distal inflation lumen 114. This lumen 114 extends from the transition interface 106 to the inflatable member 116 and is designed to carry inflation media (such as saline or contrast solution) to and from the inflatable member 116, allowing for its expansion and contraction during medical procedures. As noted above, in certain variations the inflatable member 116 may be omitted, in which case the inflation lumen 114 can also optionally be omitted.

The proximal shaft 102, which may remain at least partially outside the patient's body during use, is primarily composed of a tubular body 124. This tubular body 124 defines a proximal inflation lumen 115 that extends throughout its length. The proximal inflation lumen 115 is designed to connect with an inflation device (not shown) at the proximal end of the catheter and to carry inflation media to the distal inflation lumen 114.

The proximal shaft 102 includes a reduced-diameter distal end portion 120. In some embodiments, this portion is a skived portion, where a section of the tubular body 124 has been cut away to create a smaller profile (e.g., having a semi-annular cross-sectional shape). This skived portion 120 is configured to be slidably inserted into a receptacle 110 of the distal shaft 104 at the transition interface 106.

The receptacle 110 is a specially shaped cavity within the proximal end of the distal shaft 104, designed to receive and securely hold the skived portion 120 of the proximal shaft 102. This creates an interlocking connection between the two shafts, which maintains the structural integrity of the catheter during use. The interlocking nature of this connection helps to transfer pushing forces from the proximal shaft to the distal shaft, improving the overall pushability and control of the catheter.

When fully assembled, the proximal inflation lumen 115 of the proximal shaft 102 aligns and communicates with the distal inflation lumen 114 of the distal shaft 104. This alignment creates a continuous pathway for inflation media to flow from the inflation device at the proximal end all the way to the inflatable member 116 at the distal end.

The skived portion 120 of the proximal shaft 102 may have a semi-annular cross-section. This shape complements the interior geometry of the receptacle 110, allowing for a secure fit while still maintaining an open path for the guidewire to exit through the proximal opening 108 and enter the guidewire lumen 112. This design utilizes the limited space within the catheter's cross-section to accommodate both the inflation lumen and the guidewire path.

To enhance the performance of the catheter, particularly its ability to navigate through tortuous blood vessels, the proximal shaft 102 may include a series of flexibility-enhancing cuts 122 along its length. These cuts, which may be created using laser cutting or other precision manufacturing techniques, are strategically placed to increase the flexibility of the proximal shaft 102. However, they are designed in such a way as to maintain the shaft's pushability and resistance to kinking. This balance of flexibility and pushability facilitates effective delivery of the catheter to the target treatment site, especially in challenging vascular anatomies.

The transition interface 106 is configured to provide a smooth transition in flexibility between the relatively stiffer proximal shaft 102 and the more flexible distal shaft 104. This gradual transition helps to prevent kinking or buckling of the catheter at this junction point during navigation through curved vessels. Additionally, the interface maintains the separation between the inflation lumen and the guidewire path while also ensuring a secure connection between the two shafts.

This interlocking transition interface design aims to address several challenges in catheter engineering: it provides a smooth transition in flexibility between the proximal and distal shafts, enables efficient transfer of push forces from the proximal shaft to the distal shaft, maintains separate lumens for inflation media and guidewire passage, and enables the rapid-exchange functionality of the catheter. These features can contribute to a catheter that is both effective in delivering treatments and user-friendly for the operating physician.

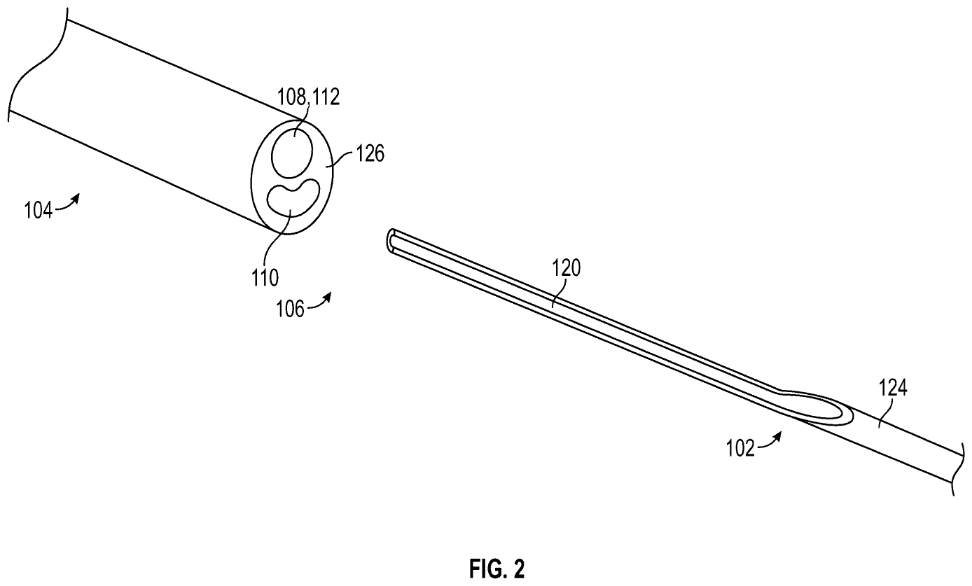

FIG. 2 is a perspective exploded view of a portion of the catheter 100, illustrating the transition interface 106 between the proximal shaft 102 and the distal shaft 104. As noted previously, the distal shaft 104 includes the receptacle 110, which is a cavity designed to receive the distal end portion 120 of the proximal shaft 102. Adjacent to the receptacle 110 is the proximal opening 108 of the guidewire lumen 112, which extends through the length of the distal shaft 104. The proximal face 126 of the distal shaft 104 is visible in this view. The proximal face 126 defines the openings for both the receptacle 110 and the guidewire lumen 112. The arrangement of these openings on the proximal face 126 facilitates the rapid exchange capability of the catheter, allowing a guidewire to exit the catheter at this point while the distal end portion 120 of the proximal shaft 102 occupies the receptacle 110.

The proximal shaft 102 is depicted with its distal end portion 120 extending towards the proximal face 126 of the distal shaft 104. This distal end portion 120 has a reduced diameter compared to the main body of the proximal shaft 102, creating a tapered or skived section. The shape and dimensions of the distal end portion 120 are configured to correspond with the internal geometry of the receptacle 110 in the distal shaft 104. In various examples, the tapered section can taper distally such that an arc length of the tapered portion is smaller in a distalmost segment of the tapered portion than at a proximalmost segment of the tapered portion.

The tubular body 124 of the proximal shaft 102 is shown to have a larger diameter than the distal end portion 120. This change in diameter creates a shoulder or transition zone between the main body of the proximal shaft 102 and its distal end portion 120. This transition may serve to limit the insertion depth of the proximal shaft 102 into the distal shaft 104, ensuring proper alignment of the components when assembled.

In the assembled configuration, the distal end portion 120 of the proximal shaft 102 would be slidably inserted into the receptacle 110 of the distal shaft 104. This insertion would create a secure connection between the two shafts while maintaining separate pathways for inflation media and guidewire passage. This configuration illustrates the interlocking nature of the transition interface 106, which is designed to provide a smooth transition between the proximal and distal shafts, facilitate the transfer of forces along the length of the catheter, and maintain the integrity of the separate lumens within the catheter assembly.

In various implementations, the catheter dimensions can be selected depending on the target anatomy. For instance, for a catheter configured for neurovascular procedures, the catheter dimensions can be scaled to navigate the small and tortuous vessels of the cerebral vasculature. The overall length of the catheter may range from about 80 cm to 180 cm to reach distal cerebral arteries from a femoral access point. The proximal shaft may have an outer diameter of approximately 0.025 inches to 0.040 inches (0.635 mm to 1.016 mm), while the distal shaft may have a slightly smaller outer diameter, perhaps ranging from 0.021 inches to 0.035 inches (0.533 mm to 0.889 mm) to enhance flexibility. In some implementations, particularly for applications outside of the neuroanatomy, the proximal shaft and/or the distal shaft can have a larger outer diameter, for instance up to about 0.079 inches (2 mm) or 0.092 (2.33 mm). The reduced-diameter distal end portion of the proximal shaft might measure about 0.018 inches to 0.030 inches (0.457 mm to 0.762 mm) in outer diameter to fit within the receptacle of the distal shaft. The guidewire lumen may accommodate guidewires of 0.010 inches to 0.014 inches (0.254 mm to 0.356 mm) in diameter, common in neurovascular interventions. The inflatable member near the distal tip could have a range of diameters from 1.5 mm to 4.5 mm when expanded, depending on the target vessel size. These dimensions are examples and may be adjusted based on specific design requirements and intended use within the neurovasculature or other target vessel.

With respect to manufacturing, suitable materials and fabrication techniques can be selected for each component. The proximal shaft can be constructed from materials such as nitinol or stainless steel, which provide a combination of strength and flexibility. This proximal shaft can be shaped using techniques such as laser cutting, drilling, or grinding to form the skived portion (optionally including engagement features such as lateral projections or prongs) and spiral cuts that enhance flexibility while maintaining pushability. For the distal shaft, softer and more pliable materials like PEBAX, nylon, or polyurethanes may be employed to provide the necessary flexibility for navigating tortuous vasculature. To assemble the catheter, the distal portion of the proximal shaft can be slidably inserted into the receptacle of the distal shaft. In some embodiments, the engagement features (e.g., lateral projections) can serve as mechanical stops that ensure a precise insertion depth of the distal portion of the proximal shaft into the receptacle. A surrounding transition shaft may be placed over this junction, with a mandrel temporarily inserted to maintain the patency of the guidewire lumen. This assembly can then be subjected to a heat-shrinking process, which bonds the components together without the need for a separate welding step. This method provides a secure connection while maintaining alignment of the proximal and distal inflation lumens, allowing for unimpeded fluid communication throughout the catheter.

### II. EXAMPLE ENGAGEMENT FEATURES FOR INTERLOCKING TRANSITION INTERFACES

In various implementations, the proximal shaft of the catheter can incorporate a variety of engagement features to facilitate a secure and functional interlocking connection with the distal shaft at the transition interface. These features may include, but are not limited to, lateral projections extending radially from the shaft, forked prongs that can be radially compressed, tapered sections for gradual insertion, ridges or grooves for increased friction, or expandable elements that deploy after insertion. In some examples, the engagement feature includes a ball bearing or other similar structure attached (e.g., welded, soldered, etc.) to the shaft. The engagement features can be designed in various shapes, sizes, and configurations to optimize the connection strength, flexibility, and case of assembly. They may be used individually or in combination, depending on the specific requirements of the catheter system. For example, a design might employ lateral projections to act as a depth stop, while also incorporating compressible prongs for radial engagement within the receptacle of the distal shaft. The selection and arrangement of these engagement features aim to create a transition interface that balances secure attachment, appropriate flexibility, and efficient transfer of forces between the proximal and distal shafts, while maintaining the necessary lumen configurations for the catheter's functionality.

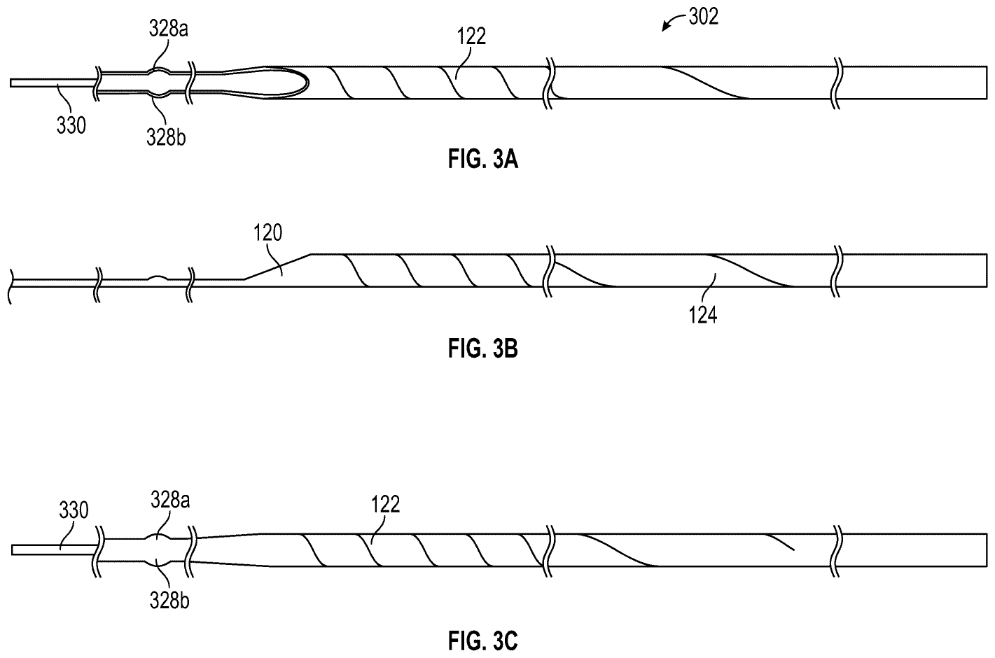

FIGS. 3A-3C are top, side, and bottom views, respectively, of an example embodiment of a proximal shaft 302 for a rapid-exchange catheter. The proximal shaft 302 has a tubular body 124 that extends along most of its length. At the distal end of the proximal shaft 302, there is a reduced-diameter distal end portion 120. This distal end portion 120 includes a tapering distal portion 330, which narrows towards the distal tip of the shaft.

Two lateral projections 328a and 328b extend outward from the distal end portion 120. These projections, which may also be referred to as wings, extend laterally (and/or radially or circumferentially) away from the centerline of the distal portion 120. The lateral projections 328a and 328b may be positioned near the proximal end of the tapering distal portion 330 such that the majority of the tapering distal portion 330 can be inserted within the receptacle 110 of the distal shaft 104 before the projections 328a and 328b abut the distal shaft 104. These projections can be configured to abut the proximal face of a corresponding distal shaft (not shown in these figures) when the proximal shaft 302 is inserted into the distal shaft. This abutment serves to lock the two shafts into place, creating a secure connection at the transition interface.

The tapering distal portion 330 is configured to facilitate insertion into a receptacle of the distal shaft and to provide a gradual transition in flexibility between the proximal and distal shafts. The lateral projections 328a and 328b, in conjunction with the tapering distal portion 330, create a specialized geometry that allows for both secure locking and controlled insertion depth when the proximal shaft 302 is assembled with its corresponding distal shaft. Additionally, this tapering distal portion 330 can facilitate the longitudinal transition of stiffness along the length of the device, thereby reducing the risk of abrupt transitions that can lead to kinking.

The lateral projections 328 can be implemented in various shapes, geometries, orientations, and configurations to optimize the interlocking mechanism and overall catheter performance. These projections may be symmetrical or asymmetrical, and can take forms such as rounded bumps, angular protrusions, or elongated fins. The orientation of the projections can vary, with some designs featuring radially outward extensions, while others may angle proximally or distally to enhance engagement with the distal shaft. In some implementations, the projections may have a tapered profile to facilitate initial insertion and lock more securely when fully engaged. The number of projections can also differ, ranging from a single projection to multiple projections arranged circumferentially around the shaft. Additionally, the size and thickness of the projections can be adjusted to balance the strength of the interlocking mechanism with the desired flexibility of the transition zone. Some designs may incorporate flexible or compressible materials for the projections to allow for a snug fit while maintaining the ability to navigate tortuous vasculature. The specific choice of projection design can be tailored to meet particular clinical needs, manufacturing considerations, and overall catheter performance requirements.

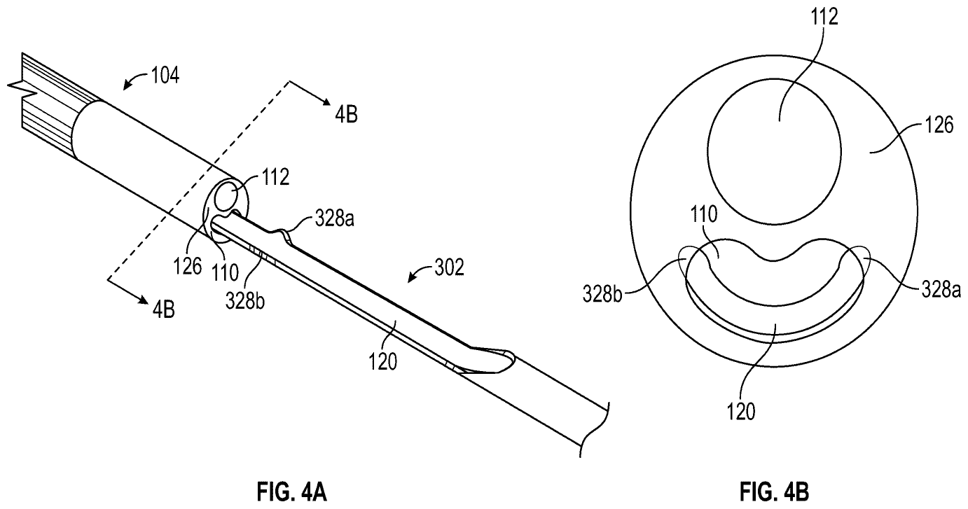

FIG. 4A is a perspective view of a catheter at the transition interface during assembly according to some embodiments of the present technology. FIG. 4B is a cross-sectional view of the catheter at the transition interface along the line 4B-4B shown in FIG. 4A. As seen in FIG. 4A, the reduced-diameter distal end portion 120 of the proximal shaft 302 is configured to be aligned with an inserted into the receptacle 110 of the distal shaft 104. The tapering distal portion of the proximal shaft 302 facilitates the insertion process. The lateral projections 328a and 328b extend outward from the distal end portion 120 of the proximal shaft 302. These projections are positioned to abut the proximal face 126 of the distal shaft 104 upon full insertion. This abutment serves to limit the insertion depth and provide a mechanical stop, ensuring proper alignment of the two shafts.

The proximal face 126 of the distal shaft 104 defines the opening of the receptacle 110 into which the proximal shaft 302 is being inserted. Adjacent to the receptacle 110, the proximal opening of the guidewire lumen 112 is disposed adjacent to the receptacle 110. As noted above, this proximal opening enables the rapid-exchange functionality of the catheter.

FIG. 4B provides a cross-sectional view of the assembly, cut along the plane indicated in FIG. 4A. This view reveals the internal structure of the transition interface 106. The reduced-diameter distal end portion 120 of the proximal shaft 302 is shown fully inserted into the receptacle 110 of the distal shaft 104. The cross-sectional shape of the distal end portion 120 appears semi-circular or crescent-shaped, complementing the interior geometry of the receptacle 110. The lateral projections 328a and 328b are shown in contact with the proximal face 126 of the distal shaft 104. This contact point illustrates how the projections act as a stop mechanism, preventing over-insertion of the proximal shaft 302 into the distal shaft 104.

The cross-sectional view shows the guidewire lumen 112 within the distal shaft 104. The lumen appears as a circular opening, separate from the space occupied by the inserted proximal shaft 302. This configuration maintains a dedicated pathway for a guidewire to pass through the transition interface. The space between the inserted distal end portion 120 of the proximal shaft 302 and the walls of the receptacle 110 forms a continuation of the inflation lumen. This crescent-shaped space allows for the passage of inflation media from the proximal shaft to the distal shaft.

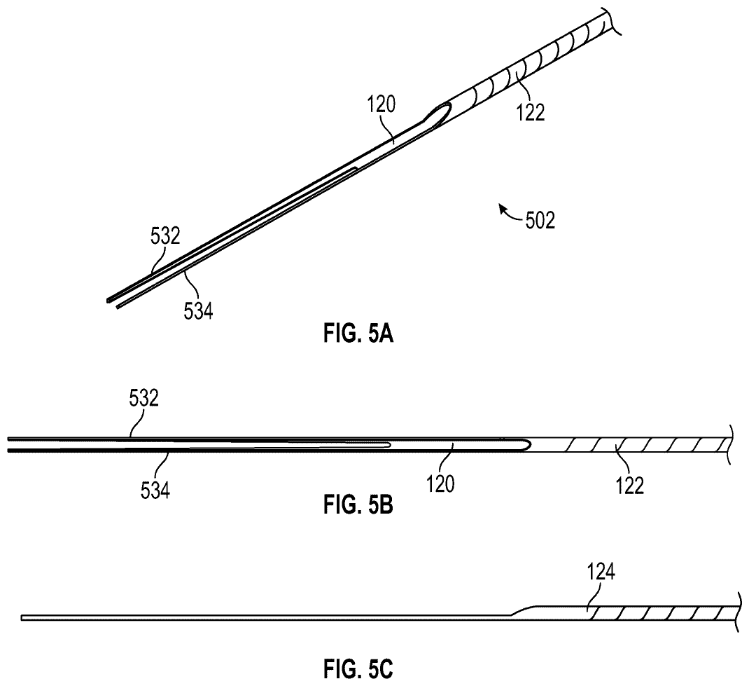

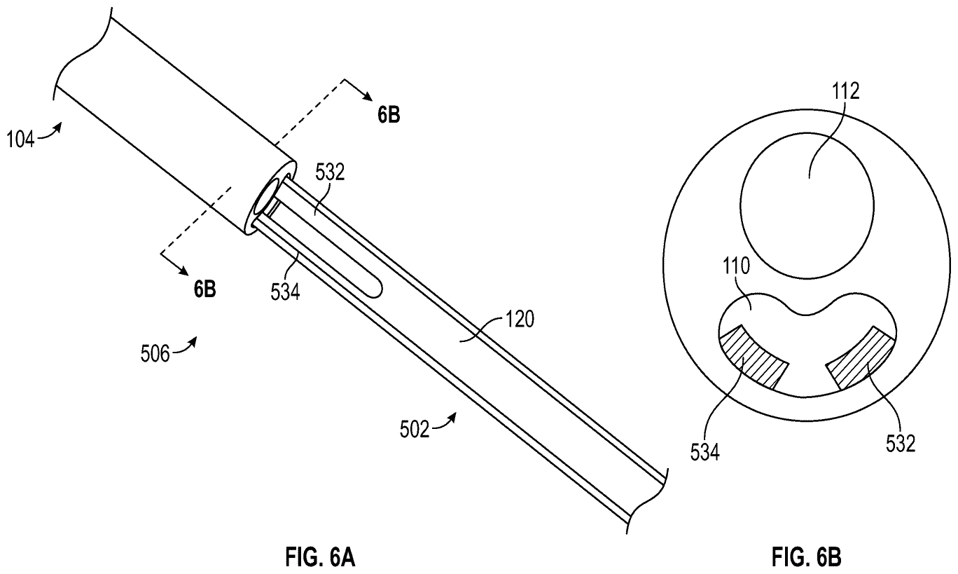

FIGS. 5A-5C and 6A-6B illustrate another embodiment of a proximal shaft 502 for a rapid-exchange catheter, featuring a forked design at its distal end portion 120. FIG. 5A shows a perspective view of the proximal shaft 502. The shaft has a tubular body 124 that extends along most of its length. At the distal end, the shaft transitions into a reduced-diameter distal end portion 120. This distal end portion 120 is characterized by a forked design, comprising a first prong 532 and a second prong 534. These prongs are separated by a longitudinal split, creating a fork-like structure.

FIG. 5B presents a top view of the proximal shaft 502. This view shows the parallel arrangement of the first prong 532 and second prong 534. The prongs extend distally from the main body of the shaft, maintaining a consistent width along their length. FIG. 5C offers a side view of the proximal shaft 502. From this angle, the transition from the tubular body 124 to the reduced-diameter distal end portion 120 is evident. The forked structure of the distal end portion 120 is visible in FIGS. 5A and 5B, showing how the prongs 532 and 534 can be formed from the same material as the shaft body. Flexibility-enhancing cuts 122 can be observed along the length of the tubular body 124. These cuts can be arranged in a pattern designed to increase the flexibility of the proximal shaft 502 while maintaining its structural integrity and pushability.

FIG. 6A presents a perspective view of the proximal shaft 502 as it is being inserted into the distal shaft 104. The forked distal end portion 120, comprising prongs 532 and 534, is shown partially inserted into the receptacle 110 of the distal shaft 104. FIG. 6B provides a cross-sectional view of the assembly, cut along the plane indicated in FIG. 6A. This view reveals the internal structure of the transition interface. The first prong 532 and second prong 534 of the proximal shaft 502 are shown fully inserted into the receptacle 110 of the distal shaft 104.

In this configuration, the prongs 532 and 534 are placed in radial compression when inserted into the receptacle 110. This compression causes the prongs to exert a lateral or radial force against the inner walls of the receptacle 110. This outward force serves to lock the prongs of the distal end portion 120 into place relative to the distal shaft 104, creating a secure connection at the transition interface. The space between the inserted prongs 532 and 534 and the walls of the receptacle 110 forms a continuation of the inflation lumen. This arrangement allows for the passage of inflation media from the proximal shaft to the distal shaft while maintaining the interlocking connection provided by the compressed prongs.

This forked design of the proximal shaft 502 illustrates an alternative approach to creating an interlocking transition interface in a rapid-exchange catheter system. The radial compression of the prongs within the receptacle can provide a secure assembly and controlled insertion, while potentially offering different flexibility characteristics compared to the wing-style projections described above.

The prongs of the forked design can be implemented in various shapes, geometries, orientations, and configurations to optimize the interlocking mechanism and overall catheter performance. The prongs may have uniform or varying widths along their length, and their cross-sectional shapes can range from rectangular to rounded or tapered profiles. In some implementations, the prongs may have an outward bias, naturally splaying apart when unconfined, to enhance their engagement with the receptacle walls. Alternatively, they may be designed with an inward bias to facilitate initial insertion. The length of the prongs can be adjusted to balance insertion depth with engagement strength. Some designs may incorporate ridges, barbs, or textured surfaces on the outer faces of the prongs to increase friction and improve locking capability. The number of prongs can vary, with some designs featuring more than two prongs arranged radially. The split between the prongs may extend partially or fully to the base of the distal end portion, affecting the flexibility and compression characteristics of the forked structure. Additionally, the prongs may be designed with different levels of flexibility along their length, for instance incorporating areas of reduced thickness or additional cuts to fine-tune their mechanical properties. The specific prong design can be tailored to meet particular clinical needs, manufacturing considerations, and overall catheter performance requirements.

The lateral projections and prongs, as well as other types of engagement features, can be combined or used individually to create versatile and effective interlocking mechanisms for the transition interface. For instance, a design might incorporate both lateral projections near the base of the distal end portion to act as a depth stop, and forked prongs at the tip for radial engagement. Alternatively, a single prong could be combined with one lateral projection to provide both longitudinal and rotational alignment. Other engagement features that could be integrated include helical ridges for a screw-like connection, circumferential grooves that mate with corresponding ridges in the receptacle, or expandable segments that deploy once inserted. These features could be used in various combinations—for example, a design might use prongs with small lateral projections at their tips, or incorporate a tapered section with both helical ridges and lateral projections. The choice and combination of engagement features can be tailored to address specific performance requirements such as case of assembly, connection strength, flexibility, and resistance to various types of mechanical stress. By combining different engagement features, designers can create transition interfaces that optimize the balance between secure connection, flexibility, and ease of manufacture for different catheter applications.

### III. CONCLUSION

Although many of the embodiments are described above with respect to systems, devices, and methods for treating vessels in the brain, the technology is applicable to other applications and/or other approaches, such as vessels elsewhere in the body. Moreover, other embodiments in addition to those described herein are within the scope of the technology. Additionally, several other embodiments of the technology can have different configurations, components, or procedures than those described herein. A person of ordinary skill in the art, therefore, will accordingly understand that the technology can have other embodiments with additional elements, or the technology can have other embodiments without several of the features shown and described above with reference to FIGS. 1A-6B.

The descriptions of embodiments of the technology are not intended to be exhaustive or to limit the technology to the precise form disclosed above. Where the context permits, singular or plural terms may also include the plural or singular term, respectively. Although specific embodiments of, and examples for, the technology are described above for illustrative purposes, various equivalent modifications are possible within the scope of the technology, as those skilled in the relevant art will recognize. For example, while steps are presented in a given order, alternative embodiments may perform steps in a different order. The various embodiments described herein may also be combined to provide further embodiments.

As used herein, the terms “generally,” “substantially,” “about,” and similar terms are used as terms of approximation and not as terms of degree, and are intended to account for the inherent embodiments in measured or calculated values that would be recognized by those of ordinary skill in the art.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with certain embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A catheter comprising:
a proximal shaft comprising:
a tubular body defining a proximal inflation lumen therethrough; and
a skived portion at a distal end region of the tubular body, the skived portion having first and second prongs; and
a distal shaft carrying an inflatable member thereon, the distal shaft comprising:
a distal inflation lumen having a distal end portion in fluid communication with the inflatable member;
a guidewire lumen extending therethrough; and
a transition interface including:
a proximal opening of the guidewire lumen into which a guidewire can be slidably inserted; and
a receptacle through which the skived portion of the proximal shaft tubular body is disposed such that the first and second prongs are placed in compression along a lateral direction and the prongs are interlocked with the receptacle, wherein the proximal inflation lumen is in fluid communication with the distal inflation lumen.

2. The catheter of claim 1, wherein the skived portion is integrally formed with the tubular body.

3. The catheter of claim 2, wherein each of the first and second prongs of the skived portion has a semi-annular cross-section.

4. The catheter of claim 3, wherein the skived portion defines a distal taper such that an arc length of the skived portion is smaller in a distalmost segment of the skived portion than at a proximalmost segment of the skived portion.

5. The catheter of claim 4, wherein the skived portion further comprises a lateral projection configured to abut a proximal face of the transition interface.

6. The catheter of claim 5, wherein the tubular body comprises one or more flexibility-enhancing cuts along its length.

7. The catheter of claim 6, further comprising a transition shaft having a distal end coupled to the distal shaft, the transition shaft circumferentially surrounding at least the skived portion of the proximal shaft tubular body.

8. A catheter comprising:
a proximal shaft comprising:
a tubular body defining a proximal inflation lumen therethrough;
a skived portion at a distal end region of the tubular body; and
a lateral projection disposed along the skived portion; and
a distal shaft carrying an inflatable member thereon, the distal shaft comprising:
a distal inflation lumen having a distal end portion in fluid communication with the inflatable member;
a guidewire lumen extending therethrough; and
a transition interface including a proximal face that defines:
a proximal opening of the guidewire lumen into which a guidewire can be slidably inserted; and
a receptacle through which the skived portion of the proximal shaft tubular body is inserted such that the lateral projection disposed along the skived portion abuts the proximal face, wherein the proximal inflation lumen is in fluid communication with the distal inflation lumen.

9. The catheter of claim 8, wherein the skived portion is integrally formed with the tubular body.

10. The catheter of claim 9, wherein the skived portion has a semi-annular cross-section.

11. The catheter of claim 10, wherein the skived portion defines a distal taper such that an arc length of the skived portion is smaller in a distalmost segment of the skived portion than at a proximalmost segment of the skived portion.

12. The catheter of claim 11, wherein the skived portion further comprises first and second prongs.

13. The catheter of claim 12, wherein the lateral projection is a first lateral projection extending along a first circumferential direction, the skived portion further comprising a second lateral projection extending along a second, opposite circumferential direction.

14. The catheter of claim 13, wherein the lateral projection comprises a wing feature.

15. The catheter of claim 14, wherein the proximal inflation lumen is in fluid communication with the distal inflation lumen via the receptacle.

16. A catheter comprising:
an elongate body having a distal end portion carrying an interventional element thereon and a proximal end portion, the elongate body comprising:
a distal shaft having a receptacle; and
a proximal shaft having a semi-tubular portion that is inserted into the distal shaft receptacle, wherein the semi-tubular portion includes an engagement feature that limits movement of the semi-tubular portion with respect to the distal shaft receptacle;
an exchange port along an intermediate portion of the elongate body; and
a guidewire lumen extending from the exchange port to the distal end portion of the elongate body.

17. The catheter of claim 16, wherein the interventional element comprises an inflatable member, the catheter further comprising an inflation lumen extending from the elongate body proximal end portion to the elongate body distal end portion, wherein the inflation lumen is in fluid communication with the inflatable member.

18. The catheter of claim 17, wherein the exchange port is disposed at the interface between the distal shaft and the proximal shaft.

19. The catheter of claim 18, wherein the engagement feature comprises a first and second prongs along the semi-tubular portion.

20. The catheter of claim 19, wherein the engagement feature comprises a lateral projection disposed along the semi-tubular portion.
